(19) United States
(12) Patent Application Publication
(10) Pub. No.: US 2024/0409490 A1
(43) Pub. Date: Dec. 12, 2024

(54) SYNTHETIC METHOD AND CATALYST OF 3-(3-OXO-2-PENTYL)CYCLOPENTYL DIMETHYL MALONATE

(71) Applicants: ZHEJIANG XINHUA CHEMICAL CO., LTD., Jiande (CN); NINGXIA XINHUA CHEMICAL CO., LTD., Yinchuan (CN)

(72) Inventors: Jian Zhu, Jiande (CN); Sibin Ying, Jiande (CN); Jingheng Wu, Jiande (CN); Dengyu Ying, Jiande (CN); Gongyu Luo, Jiande (CN); Yuanyuan Qiao, Jiande (CN)

(21) Appl. No.: 18/288,957

(22) PCT Filed: Dec. 27, 2022
(86) PCT No.: PCT/CN2022/142370

§ 371 Date: Oct. 30, 2023

(30) Foreign Application Priority Data
May 11, 2022 (CN) 202210513694.3

(51) Int. Cl. C07C 67/347; B01J 31/02

(52) U.S. Cl. CPC C07C 67/347; B01J 31/0282; B01J 31/0284; B01J 31/0298; B01J 2231/32

(57) ABSTRACT

A synthetic method and catalyst for preparing 3-(3-oxo-2-pentyl)cyclopentyl dimethyl malonate are disclosed. The synthetic method uses 2-pentyl-2-cyclopentenone and dimethyl malonate as raw materials, and reacts them in the presence of a catalyst to prepare 3-(3-oxo-2-pentyl)cyclopentyl dimethyl malonate, the catalyst is a basic ionic liquid, the pH value of the basic ionic liquid is greater than or equal to 10, and the catalyst is prepared by a method comprising the steps of: mixing the nitrogen-containing heterocyclic compound with an aliphatic carboxylate or hydroxyl aliphatic carboxylate or fluorophosphate under stirring. The synthetic method is environmentally friendly, stable in reaction and low-cost, and the conversion of 2-pentyl-2-cyclopentenone is significantly improved due to the use of the above-mentioned basic ionic liquid as catalyst.

### CROSS-REFERENCE TO RELATED APPLICATION

This application is the U.S. National Stage of PCT App. Serial No. PCT/CN2022/142370, having an International Filing Date of Dec. 27, 2022, which claims the benefit of priority to Chinese Patent Application No. 2022105136943 filed on May 11, 2022, and the entire disclosure of both are hereby incorporated by reference.

### FIELD OF THE INVENTION

The present disclosure relates to a synthetic method and catalyst for preparing 3-(3-oxo-2-pentyl)cyclopentyl dimethyl malonate.

### BACKGROUND OF THE INVENTION

3-oxo-2-pentyl cyclopentyl methyl acetate, also known as methyl dihydrojasmonate (MDJ), belongs to jasmonate and is an important artificial flavor. MDJ is a colorless or yellowish transparent liquid with relatively stable chemical properties, and has the advantages of slow volatilization, long fragrance retention time, and will not cause discoloration when used for flavoring, and it is often used to prepare oriental flavors such as jasmine, lily of the valley, tuberosa, etc.

In industry, cyclopentanone, n-pentanal and dimethyl malonate are mainly used as raw materials to synthesize methyl dihydrojasmonate. The specific process is as follows: 1) Cyclopentanone and n-pentanal undergo condensation dehydration reaction to produce 2-pentylidene cyclopentan-1-one; 2) 2-pentylidene cyclopentan-1-one undergoes isomerization reaction to produce 2-pentyl-2-cyclopentenone; 3) 2-pentyl-2-cyclopentenone and dimethyl malonate undergo a Michael Addition reaction to produce 3-(3-oxo-2-pentyl)cyclopentyl dimethyl malonate; 4) 3-(3-oxo-2-amyl)cyclopentyl dimethyl malonate is hydrolyzed and decarboxylated to give methyl dihydrojasmonate.

For the Michael Addition reaction between 2-pentyl-2-cyclopentenone and dimethyl malonate to produce 3-(3-oxo-2-pentyl)cyclopentyl dimethyl malonate, sodium methoxide is mainly used as the catalyst in industry at present, for example, Chinese patent CN101429122B discloses a decarboxylation method in the synthesis of methyl dihydrojasmonate, in which methyl dihydrojasmonate is obtained by addition and decarboxylation of 2-pentyl cyclopentenone and dimethyl malonate under the catalysis of sodium methoxide in methanol solution. However, the use of sodium methoxide as a catalyst has the following problems: first, sodium methoxide cannot be recycled and reused, acid quenching is required at the end of the reaction, and a large amount of saturated sodium bicarbonate aqueous solution and salt solution are required to wash the organic phase, which will form a large amount of salt-containing wastewater, leading to unfriendly environment waste; secondly, sodium methoxide is particularly sensitive to water during use, a small amount of water will cause the decomposition of sodium methoxide, leading to an unstable reaction; finally, due to the high viscosity of sodium methoxide, a large amount of methanol needs to be added as the solvent in the reaction process, and the addition of solvent will not only increase the separation energy consumption, but also increase the handling load of the three wastes.

### SUMMARY OF THE INVENTION

In view of the shortcomings and deficiencies of in the art, the present disclosure provides an improved synthetic method of 3-(3-oxo-2-pentyl)cyclopentyl dimethyl malonate, which is environmentally friendly, stable in reaction and low in production cost.

To achieve the above purposes, a technical solution employed by the present disclosure is as follows:

A synthetic method for preparing 3-(3-oxo-2-pentyl)cyclopentyl dimethyl malonate, which uses 2-pentyl-2-cyclopentenone and dimethyl malonate as raw materials, and reacts them in the presence of a catalyst to prepare 3-(3-oxo-2-pentyl)cyclopentyl dimethyl malonate, the catalyst is a basic ionic liquid, and the pH value of the basic ionic liquid is greater than or equal to 10; and the catalyst is prepared by a preparation method comprising the following steps of: mixing the nitrogen-containing heterocyclic compound with an aliphatic carboxylate or hydroxyl aliphatic carboxylate or fluorophosphate under stirring.

In some implementations of the present disclosure, the aliphatic carboxylate is selected from the group consisting of the salt of R₁COOH or HOOCR₂COOH, and the hydroxyl aliphatic carboxylate is the salt of OHR₃COOH, and the fluorophosphates is selected from the group consisting of trifluorophosphate, tetrafluorophosphate, hexafluorophosphate, and combinations thereof, wherein, R₁ is selected from the group consisting of C1-C8 alkyl, R₂ is selected from the group consisting of a single bond and C1-C7 alkylidene, and R₃ is selected from the group consisting of C1-C5 alkylidene.[0009] In some implementations of the present disclosure, the pH value of the basic ionic liquid is 12-14.

In some implementations of the present disclosure, the nitrogen-containing heterocyclic compound is selected from the group consisting of 1,8-diazabicyclo[5.4.0]undec-7-ene

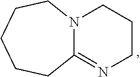 [SMILES: C1CCC2=NCCCN2CC1]

4-dimethylaminopyridine

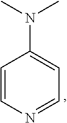

1,5-diazabicyclo[4.3.0]non-5-ene

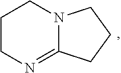

and combinations thereof.

In some implementations of the present disclosure, the aliphatic carboxylate is selected from the group consisting of acetate, propionate or oxalate; and the hydroxyl aliphatic carboxylate is selected from the group consisting of glycolate, hydroxypropionate or hydroxybutyrate.

For the Michael Addition reaction between 2-pentyl-2-cyclopentenone and dimethyl malonate under the action of the basic ionic liquid, this generates 3-(3-oxo-2-pentyl)cyclopentyl dimethyl malonate, with the reaction formula as follows:

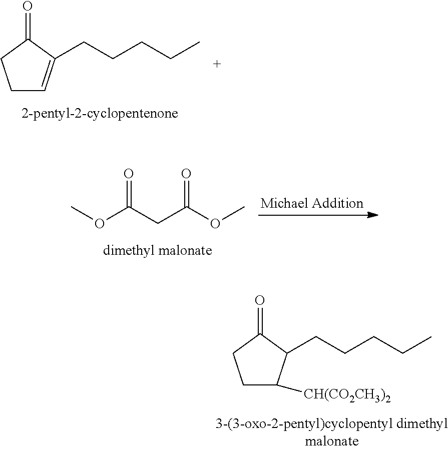

The authors of the present disclosure found that when a strong basic ionic liquid with a pH value greater than or equal to 10 formed by nitrogen-containing heterocyclic compound is used as the catalyst for Michael Addition reaction between 2-pentyl-2-cyclopentenone and dimethyl malonate to generate 3-(3-oxo-2-pentyl)cyclopentyl dimethyl malonate, the reaction does not need solvents, and the reaction can be environmentally friendly, stable and low in production cost.

In some implementations of the present disclosure, the reaction is carried out in the presence of a monodentate phosphine ligand. The presence of the monodentate phosphine ligand can further improve the conversion of 2-pentyl-2-cyclopentenone.

Further, the monodentate phosphine ligand is selected from the group consisting of triphenylphosphine, [4-(N,N-dimethylamino)phenyl]di(tert-butyl)phosphine, diphenyl-2-pyridylphosphine, [4-(dimethylamino)phenyl]diphenylphosphine, combinations thereof.

In an implementation of the present disclosure, the molar ratio of the monodentate phosphine ligand to the catalyst is 1:(1-50).

In some implementations of the present disclosure, the molar ratio of 2-pentyl-2-cyclopentenone to dimethyl malonate is 1:(0.5-5).

In some implementations of the present disclosure, the mass ratio of the catalyst to dimethyl malonate is 1:(10-50).

In some implementations of the present disclosure, the synthetic method comprises steps of: mixing dimethyl malonate and the catalyst, or mixing dimethyl malonate, the catalyst and the monodentate phosphine ligand to obtain a mixture, adding 2-pentyl-2-cyclopentenone dropwise into the mixture, and stirring at a constant temperature to continue the reaction after the addition.

In further implementations, the temperature of the mixture during addition is −10 ˜30° C., and the addition time is 1˜10 h.

In further implementations, the constant temperature is −10˜50° C., and the time for the constant temperature is 1˜30 h.

In embodiment further implementation, after the addition is completed, the reaction system is cooled to −5° C., and then stirred to continue the reaction at this temperature.

In some implementations of the present disclosure, the synthetic method further comprises steps of adding water to the reaction system for static stratification after the reaction is completed.

After static stratification, the upper layer is an organic layer and the lower layer is a water layer, the water in the water layer can be removed by distillation to recover the basic ionic liquid catalyst.

The present disclosure also provides the above-mentioned catalyst.

The present disclosure further provides a process for the preparation of the above-mentioned catalyst, which comprises steps of: mixing the nitrogen-containing heterocyclic compound with an aliphatic carboxylate or hydroxyl aliphatic carboxylate or fluorophosphate under stirring; the aliphatic carboxylate is selected from the group consisting of the salt of R₁COOH or HOOCR₂COOH, and the hydroxyl aliphatic carboxylate is the salt of OHR₃COOH, and the fluorophosphates is selected from the group consisting of trifluorophosphate, tetrafluorophosphate, hexafluorophosphate, and combinations thereof, wherein, R₁ is selected from the group consisting of C1-C8 alkyl, R₂ is selected from the group consisting of a single bond and C1-C7 alkylidene, and R₃ is selected from the group consisting of C1-C5 alkylidene.

Further, the mixing temperature is 20-80° C., and the mixing time is 4-24 h.

The above-mentioned catalyst provides a yellowish transparent liquid.

The present disclosure has the following advantages over the prior art:

The mixing between the nitrogen-containing heterocyclic compound with the aliphatic carboxylate or hydroxyl aliphatic carboxylate or fluorophosphate is used to form a strong basic ionic liquid with a pH value greater than or equal to 10, and when it is used to as catalyst for the synthetic method, the synthetic method does not need solvents, the reaction conditions are mild, and the conversion rate of 2-pentyl-2-cyclopentenone can be improved.

The catalyst of the present disclosure is simple to prepare, easy to recycle and reuse, and remains basically unchanged in activity after being applied multiple times, and has good stability.

The synthetic method of the present disclosure does not require solvents, and only water is used for catalyst recycle, resulting in low discharge of three wastes, low cost, and environmental friendliness.

By adding the monodentate phosphine ligand in the reaction system, the present disclosure can further improve the conversion rate of 2-pentyl-2-cyclopentenone, which is up to 99%.

### DETAILED DESCRIPTION OF THE PREFERRED EMBODIMENTS

The present disclosure is further described below combining with embodiments. The present disclosure should be understood to not be not limited to the embodiments below. The implementation conditions used in the embodiments herein may be further adjusted according to different requirements of specific use, and undefined implementation conditions usually are conventional conditions in the industry. The technical features involved in the various embodiments of the present disclosure may be combined with each other if they do not conflict with each other.

### Embodiment 1

### 1) Preparation of Basic Ionic Liquid

0.5 mol of 1,8-diazabicyclo[5.4.0]undec-7-ene was weighed, 1 mol of sodium acetate was placed in a beaker, and stirred at 30° C. for 6 h at a constant temperature, to give the desired ionic liquid with a pH value of 13.2.

2) Preparation of 3-(3-oxo-2-pentyl)cyclopentyl dimethyl malonate

2 g of the above ionic liquid and 50 g of dimethyl malonate were weighed into a 100 mL three necked flask, the three necked flask was placed in a 25° C. constant-temperature water bath; then 56 g of 2-pentyl-2-cyclopentenone was slowly added dropwise, the addition time was controlled to be to 6 h, and after the dropwise addition was completed, the temperature of the water bath was lowered to −5° C., and the system was stirred for 6 h at a constant temperature.

### 3) Product Analysis

After the reaction was completed, 10 g of deionized water was added, and the system was stirred thoroughly and static stratification. The upper layer was the organic phase and the lower layer was the aqueous solution of ionic liquid. The products were analyzed using an Agilent 7890 gas chromatograph, in which the column was HP-INNOWax and the detector was a TCD detector. The conversion rate and selectivity were calculated by normalization.

### Embodiment 2

This embodiment was similar to Embodiment 1, differing only in that 1,8-diazabicyclo[5.4.0]undec-7-ene was replaced by 4-dimethylaminopyridine, and the pH value of the ionic liquid was 11.7.

### Embodiment 3

This embodiment was similar to Embodiment 1, differing only in that 1,8-diazabicyclo[5.4.0]undec-7-ene was replaced by 1,5-diazabicyclo[4.3.0]non-5-ene, and the pH value of the ionic liquid was 13.4.

For Embodiments 1-3, the conversion rate of 2-pentyl-2-cyclopentenone and the selectivity of 3-(3-oxo-2-pentyl)cyclopentyl dimethyl malonate are shown in Table 1 below:

TABLE 1
Conversion rate and selectivity of Embodiments 1-3

| | | Conversion | Selectivity of |
|---|---|---|---|
| | | rate of | 3-(3-oxo-2- |
| | | 2-pentyl- | pentyl)cyclo- |
| | | 2-cyclo- | pentyl |
| | Nitrogen-containing | pentenone | dimethyl |
| Embodiments | heterocyclic compounds | (%) | malonate (%) |
| Embodiment | 1,8-diazabicyclo | 93 | 98 |
| 1 | [5.4.0]undec-7-ene | | |
| Embodiment | 4-dimethylaminopyridine | 91 | 97 |
| 2 | | | |
| Embodiment | 1,5-diazabicyclo | 93 | 96 |
| 3 | [4.3.0]non-5-ene | | |

As can be seen from Table 1, the different nitrogen-containing heterocyclic basic ionic liquids showed excellent activity, with a conversion rate of 2-pentyl-2-cyclopentenone≤90% and a selectivity of 3-(3-oxo-2-pentyl)cyclopentyl dimethyl malonate>95%.

### Embodiment 4

This embodiment was similar to Embodiment 1, differing only in that sodium acetate was replaced by potassium acetate, and the pH value of the ionic liquid was 13.3.

### Embodiment 5

This embodiment was similar to Embodiment 1, differing only in that sodium acetate was replaced by lithium acetate, and the pH value of the ionic liquid was 12.9.

For Embodiments 1, 4-5, the conversion rate of 2-pentyl-2-cyclopentenone and the selectivity of 3-(3-oxo-2-pentyl)cyclopentyl dimethyl malonate are shown in Table 2 below:

TABLE 2
Conversion rate and selectivity of Embodiments 1, 4-5

| | | Conversion | Selectivity of |
|---|---|---|---|
| | | rate of | 3-(3-oxo-2- |
| | | 2-pentyl-2- | pentyl)cyclopentyl |
| | Aliphatic | cyclopentenone | dimethyl |
| Embodiments | carboxylates | (%) | malonate (%) |
| Embodiment 1 | Sodium acetate | 93 | 98 |
| Embodiment 4 | Potassium acetate | 92 | 97 |
| Embodiment 5 | Lithium acetate | 93 | 96 |

As can be seen from Table 2, the three cations, namely sodium, potassium and lithium, resulted in similar activity of nitrogen-containing heterocyclic basic ionic liquids, with a conversion rate of 2-pentyl-2-cyclopentenone>900 and a selectivity of 3-(3-oxo-2-pentyl)cyclopentyl dimethyl malonate>950.

### Embodiments 6-13

These embodiments were similar to Embodiment 1, differing only in that sodium acetate was replaced by aliphatic carboxylates or hydroxyl aliphatic carboxylates or fluorophosphates showed in Table 3. For Embodiments 6-13, the conversion rate of 2-pentyl-2-cyclopentenone and the selectivity of 3-(3-oxo-2-pentyl)cyclopentyl dimethyl malonate are shown in Table 3 below:

TABLE 3
Conversion rate and selectivity of Embodiments 1, 6-13

| | | | Conversion | |
|---|---|---|---|---|
| | Aliphatic | | rate of | Selectivity of |
| | carboxylates | pH of | 2-pentyl- | 3-(3-oxo-2- |
| | or hydroxyl aliphatic | basic | 2-cyclo- | pentyl)cyclo- |
| | carboxylates or | ionic | pentenone | pentyl dimethyl |
| Embodiments | fluorophosphates | liquids | (%) | malonate (%) |
| Embodiment | Sodium acetate | 13.2 | 93 | 98 |
| 1 | | | | |
| Embodiment | Sodium propionate | 12.4 | 93 | 97 |
| 6 | | | | |
| Embodiment | Sodium oxalate | 11.9 | 92 | 98 |
| 7 | | | | |
| Embodiment | Sodium glycolate | 11.7 | 88 | 97 |
| 8 | | | | |
| Embodiment | Sodium | 12.4 | 90 | 97 |
| 9 | hydroxypropionate | | | |
| Embodiment | Sodium | 11.9 | 87 | 96 |
| 10 | hydroxybutyrate | | | |
| Embodiment | Sodium | 13.1 | 93 | 95 |
| 11 | trifluorophosphate | | | |
| Embodiment | Sodium | 13.1 | 92 | 93 |
| 12 | tetrafluorophosphate | | | |
| Embodiment | Sodium | 12.7 | 92 | 93 |
| 13 | hexafluorophosphate | | | |

As can be seen from Table 3, all of the basic ionic liquids prepared with different anions in the selected range showed excellent catalytic activity, with a conversion rate of 2-pentyl-2-cyclopentenone>88% and a selectivity of 3-(3-oxo-2-pentyl)cyclopentyl dimethyl malonate>90%.

### Embodiment 14

The aqueous solution of the ionic liquid in Embodiment 1 was rotary evaporated to remove the water, and the ionic liquid can be recycled, the reusing method being the same as in Embodiment 1. The recycle results are shown in Table 4:

TABLE 4
Comparison of the performance of recycled
basic ionic liquids prepared in Embodiment 1

| | Conversion | Selectivity of 3- |
|---|---|---|
| | rate of | (3-oxo-2- |
| | 2-pentyl-2- | pentyl)cyclopentyl |
| Recycled | cyclopentenone | dimethyl |
| times | (%) | malonate (%) |
| 1 | 93 | 98 |
| 2 | 93 | 96 |
| 3 | 94 | 98 |
| 4 | 93 | 97 |
| 5 | 92 | 98 |

As can be seen from Table 4, after being recycled for 5 times, the activity of the catalyst remains basically unchanged, indicating that the performance of the prepared basic ionic liquid is stable.

### Embodiment 15

### 2) Preparation of Basic Ionic Liquid

The preparation method was the same as in Embodiment 1.

2) Preparation of 3-(3-oxo-2-pentyl)cyclopentyl dimethyl malonate

2 g of the above ionic liquid, 0.3 g of [4-(N,N-dimethylamino)phenyl]di(tert-butyl)phosphine, and 50 g of dimethyl malonate were weighed into a 100 mL three necked flask, the three necked flask was placed in a 25° C. constant-temperature water bath; then 56 g of 2-pentyl-2-cyclopentenone was slowly added dropwise, the addition time was controlled to be to 6 h, and after the dropwise addition was completed, the temperature of the water bath was lowered to −5° C., and the system was stirred for 6 h at a constant temperature.

### 3) Product Analysis

The analysis method was the same as in Embodiment 1.

### Embodiments 16-18

These embodiments were similar to Embodiment 15, differing only in that [4-(N,N-dimethylamino)phenyl]di(tert-butyl)phosphine was replaced by triphenylphosphine, diphenyl-2-pyridylphosphine, [4-(dimethylamino)phenyl]diphenylphosphine, respectively. The catalytic performance of Embodiments 15-18 is shown in Table 5 below:

TABLE 5
Conversion rate and selectivity of Embodiments 1, 15-18

| | | | Selectivity of |
|---|---|---|---|
| | | Conversion | 3-(3-oxo-2- |
| | | rate of | pentyl) |
| | | 2-pentyl-2- | cyclopentyl |
| | Monodentate phosphine | cyclopentenone | dimethyl |
| Embodiments | ligands | (%) | malonate (%) |
| Embodiment | None | 93 | 98 |
| 1 | | | |
| Embodiment | [4-(N,N-dimethylamino) | 99 | 98 |
| 15 | phenyl]di | | |
| | (tert-butyl)phosphine | | |
| Embodiment | Triphenylphosphine | 95 | 96 |
| 16 | | | |
| Embodiment | Diphenyl-2- | 97 | 96 |
| 17 | pyridylphosphine | | |
| Embodiment | [4-(dimethylamino) | 99 | 97 |
| 18 | phenyl]diphenylphosphine | | |

As can be seen from Table 5, the addition of the monodentate phosphine ligands can increase the conversion rate of 2-pentyl-2-cyclopentenone, with a conversion rate of 2-pentyl-2-cyclopentenone>95% and a selectivity of 3-(3-oxo-2-pentyl)cyclopentyl dimethyl malonate>95%.

### Comparative Example 1

This comparative example is similar Embodiment 1, differing only in that: the 1,8-diazabicyclo[5.4.0]undec-7-ene was replaced by methylimidazole, and the pH value of the ionic liquid was 8.7.

The results showed that the conversion rate of 2-pentyl-2-cyclopentenone was 41%, and the selectivity of 3-(3-oxo-2-pentyl)cyclopentyl dimethyl malonate was 81%.

### Comparative Example 2

3) 2 g of 1,8-diazabicyclo[5.4.0]undec-7-ene and 50 g of dimethyl malonate were weighed into a 100 mL three necked flask, the three necked flask was placed in a 25° C. constant-temperature water bath; then 56 g of 2-pentyl-2-cyclopentenone was slowly added dropwise, the addition time was controlled to be to 6 h, and after the addition was completed, the temperature of the water bath was lowered to −5° C., and the system was stirred for 6 h at a constant temperature.

4) After the reaction was completed, 10 g of deionized water was added, and the system was stirred thoroughly and static stratification. The upper layer was the organic phase, and the product was analyzed by gas chromatography.

### Comparative Examples 3-4

Comparative examples 3 and 4 are similar to Embodiment 2, differing only in that: 1,8-diazabicyclo[5.4.0]undec-7-ene was replaced by 4-dimethylaminopyridine,1,5-diazabicyclo[4.3.0]non-5-ene, respectively. The catalytic activity results for Comparative examples 2-4 are shown in Table 6 below:

TABLE 6
Conversion rate and selectivity of Comparative examples 2-4

| | Conversion | Selectivity of |
|---|---|---|
| | rate of | 3-(3-oxo-2-pentyl) |
| | 2-pentyl-2- | cyclopentyl |
| Comparative | cyclopentenone | dimethyl |
| examples | (%) | malonate (%) |
| Comparative example | 44 | 97 |
| 2 | | |
| Comparative example | 39 | 97 |
| 3 | | |
| Comparative example | 43 | 93 |
| 4 | | |

As can be seen from Table 6, nitrogen-containing heterocyclic compounds can also catalyze the reaction between dimethyl malonate and 2-pentyl-2-cyclopentenone, but the conversion rate of 2-pentyl-2-cyclopentenone is significantly reduced, indicating that the conversion rate of 2-pentyl-2-cyclopentenone can be significantly improved after nitrogen-containing heterocyclic compounds form ionic liquids with aliphatic carboxylates or hydroxyl aliphatic carboxylates or fluorophosphate.

### Comparative Example 5

5) 2 g of sodium acetate, 0.3 g of [4-(N,N-dimethylamino)phenyl]di(tert-butyl)phosphine, and 50 g of dimethyl malonate were weighed into a 100 mL three necked flask, the three necked flask was placed in a 25° C. constant-temperature water bath; then 56 g of 2-pentyl-2-cyclopentenone was slowly added dropwise, the addition time was controlled to be to 6 h, and after the dropwise addition was completed, the temperature of the water bath was lowered to −5° C., and the system was stirred for 6 h at a constant temperature.

6) After the reaction was completed, 10 g of deionized water was added, and the system was stirred thoroughly and static stratification. The upper layer was an organic phase, and the analysis showed that 3-(3-oxo-2-pentyl)cyclopentyl dimethyl malonate was not detected in the reactants. These results indicated that nitrogen-containing heterocyclic compounds are the main active sites of the catalyst.

The embodiments described above are only for illustrating the technical concepts and features of the present disclosure, and are intended to make those skilled in the art being able to understand the present disclosure and thereby implement it, and should not be concluded to limit the protective scope of this disclosure. Any equivalent variations or modifications according to the spirit of the present disclosure should be covered by the protective scope of the present disclosure.

The endpoints and any values of the ranges disclosed herein are not limited to the precise ranges or values, and these ranges or values should be understood to include values close to these ranges or values. For ranges of value, between the end values of each range, between the end values of each range and individual point values, and between individual point values can be combined with each other to obtain one or more new ranges of value, and these ranges of value should be considered as specifically disclosed herein.

1. (canceled)

2. A synthetic method for preparing 3-(3-oxo-2-pentyl)cyclopentyl dimethyl malonate, comprising reacting raw materials 2-pentyl-2-cyclopentenone and dimethyl malonate in the presence of a catalyst to prepare 3-(3-oxo-2-pentyl)cyclopentyl dimethyl malonate, wherein, the catalyst is a basic ionic liquid, the pH value of the basic ionic liquid is greater than or equal to 10; and further wherein the catalyst is prepared by a method comprising: mixing the nitrogen-containing heterocyclic compound with an aliphatic carboxylate or hydroxyl aliphatic carboxylate or fluorophosphate under stirring.

3. The synthetic method according to claim 2, wherein, the pH value of the basic ionic liquid is 12-14.

4. The synthetic method according to claim 2, wherein, the nitrogen-containing heterocyclic compound is selected from the group consisting of 1,8-diazabicyclo[5.4.0]undec-7-ene, 4-dimethylaminopyridine, 1,5-diazabicyclo[4.3.0]non-5-ene, and combinations thereof.

5. The synthetic method according to claim 2, wherein, the aliphatic carboxylate is selected from the group consisting of the salt of R₁COOH or HOOCR₂COOH, and the hydroxyl aliphatic carboxylate is the salt of OHR₃COOH, and the fluorophosphates is selected from the group consisting of trifluorophosphate, tetrafluorophosphate, hexafluorophosphate, and combinations thereof; wherein, R₁ is selected from the group consisting of C1-C8 alkyl, R₂ is selected from the group consisting of a single bond, and C1-C7 alkylidene, and R₃ is selected from the group consisting of C1-C5 alkylidene.

6. The synthetic method according to claim 2, wherein, the aliphatic carboxylate is selected from the group consisting of acetate, propionate or oxalate; and the hydroxyl aliphatic carboxylate is selected from the group consisting of glycolate, hydroxypropionate or hydroxybutyrate.

7. The synthetic method according to claim 2, wherein, the reaction is carried out in the presence of a monodentate phosphine ligand.

8. The synthetic method according to claim 7, wherein, the monodentate phosphine ligand is selected from the group consisting of triphenylphosphine, [4-(N,N-dimethylamino)phenyl]di(tert-butyl)phosphine, diphenyl-2-pyridylphosphine, [4-(dimethylamino)phenyl]diphenylphosphine, combinations thereof.

9. The synthetic method according to claim 7, wherein, the molar ratio of the monodentate phosphine ligand to the catalyst is 1:(1-50).

10. The synthetic method according to claim 2, wherein, the molar ratio of 2-pentyl-2-cyclopentenone to dimethyl malonate is 1:(0.5-5); and/or, the mass ratio of the catalyst to dimethyl malonate is 1:(10-50).

11. The synthetic method according to claim 2, wherein, the synthetic method comprises steps of: mixing dimethyl malonate and the catalyst to obtain a mixture, adding 2-pentyl-2-cyclopentenone dropwise into the mixture, and stirring at a constant temperature to continue the reaction after the addition.

12. The synthetic method according to claim 11, wherein, the temperature of the mixture during addition is −10-30° C., and the addition time is 1˜10 h; and/or, the constant temperature is −10-50° C., and the time for the constant temperature is 1-30 h.

13. The synthetic method according to claim 11, wherein, the synthetic method further comprises steps of adding water to the reaction system for static stratification after the reaction is completed.

14. The catalyst according to claim 2.

15. A process for the preparation of the catalyst according to claim 14, wherein, the process comprises steps of: mixing the nitrogen-containing heterocyclic compound with the aliphatic carboxylate or hydroxyl aliphatic carboxylate or fluorophosphate under stirring; the aliphatic carboxylate is selected from the group consisting of the salt of R₁COOH or HOOCR₂COOH, and the hydroxyl aliphatic carboxylate is the salt of OHR₃COOH, and the fluorophosphates is selected from the group consisting of trifluorophosphate, tetrafluorophosphate, hexafluorophosphate, and combinations thereof, wherein, R₁ is selected from the group consisting of C1-C8 alkyl, R₂ is selected from the group consisting of a single bond and C1-C7 alkylidene, and R₃ is selected from the group consisting of C1-C5 alkylidene.

16. The process according to claim 15, the mixing temperature is 20-80° C., and the mixing time is 4-24 h.

17. The synthetic method according to claim 2, wherein, the pH value of the basic ionic liquid is 12-14, the nitrogen-containing heterocyclic compound is selected from the group consisting of 1,8-diazabicyclo[5.4.0]undec-7-ene, 4-dimethylaminopyridine, 1,5-diazabicyclo[4.3.0]non-5-ene, and combinations thereof; the aliphatic carboxylate is selected from the group consisting of acetate, propionate or oxalate; and the hydroxyl aliphatic carboxylate is selected from the group consisting of glycolate, hydroxypropionate or hydroxybutyrate; and the fluorophosphates is selected from the group consisting of trifluorophosphate, tetrafluorophosphate, hexafluorophosphate, and combinations thereof, the molar ratio of 2-pentyl-2-cyclopentenone to dimethyl malonate is 1:(0.5-5); the mass ratio of the catalyst to dimethyl malonate is 1:(10-50).

18. The synthetic method according to claim 7, wherein, the synthetic method comprises steps of: mixing dimethyl malonate, the catalyst and the monodentate phosphine ligand to obtain a mixture, adding 2-pentyl-2-cyclopentenone dropwise into the mixture, and stirring at a constant temperature to continue the reaction after the addition.
